(19) United States
(12) Patent Application Publication
(10) Pub. No.: US 2004/0048837 A1
(43) Pub. Date: Mar. 11, 2004

(54) Method for treatment of renal disease

(76) Inventor: J. Michael Lazarus, Wellesley Hills, MA (US)

Correspondence Address: Stanley J. Gradisar, Suite 4100, 1801 California Street, Denver, CO, 80202, US

(21) Appl. No.: 10/236,476

(22) Filed: Sep. 6, 2002

(51) Int. Cl. A61K 31/59

(52) U.S. Cl. 514/167

(57) ABSTRACT

A method for reducing mortality in renal failure patients such as dialysis patients by administering paricalcitol in place of calcitriol, preferably without regard to the secondary hyperparathyroidism, calcium or phosphate status of the patient.

### FIELD OF THE INVENTION

This invention relates to the treatment of renal disease using a Vitamin D analogue to enhance survival and reduce mortality.

### BACKGROUND OF THE INVENTION

Chronic renal disease, sometimes called “kidney failure,” is a serious and prevalent health problem affecting millions of individuals. At the extreme, called End State Renal Disease (or “ESRD”), these toxin build-ups can, and do, poison and kill the patient. Chronic renal disease is commonly caused by diabetes, but can also be caused by hypertension, immunologic disorders, genetic disorders, or nephrotoxic drugs.

Because the kidneys process and remove toxins and other wastes from the bloodstream, such as urea and creatinine, the result of progressive kidney disease is a build-up of these waste products. This build-up produces a variety of detrimental chemical imbalances in the patient that affect physiological and neuropsychiatric function, producing many symptoms.

End State Renal Disease is commonly treated by dialysis. In the United States alone, approximately 200,000 patients suffer from chronic renal failure to the point that they undergo dialysis. There are two principal types of dialysis: hemodialysis and peritoneal dialysis. Hemodialysis involves establishing an extracorporeal blood circuit for the patient. This is done by accessing the vascular system with a needle through a fistula or cannula, or by way of a central catheter, allowing the blood to flow through a circuit outside the body for the dialysis treatment, and replacing the blood at distant vascular system site.

The dialysis process in hemodialysis is accomplished with a hemodialysis membrane. Removed blood flows on one side of the membrane, while a desired dialysate flows on the opposite side. Osmotic pressures and concentration gradients generated across the membrane by the differential constituent concentrations between the blood and the dialysate, produce flow of undesired materials from the blood to the dialysate and flow of desired materials from the dialysate to the blood.

In practice, the hemodialysis membrane is created as a set of hollow fibers with blood flowing through the fiber lumen and dialysate flowing outside the fibers, all in a dialyzer housing. This arrangement increases the surface area of the membrane so as to increase the overall transfer rate across the membrane. Hemodialysis is a continuous process; a pump on the blood side continually renews the blood that is being treated by removing untreated blood from the patient and replacing treated blood back into the patient, and a pump on the dialysis side continually renews the dialysate by drawing new dialysate from a bag reservoir and pumping the spent dialysate into a waste bag or container.

In peritoneal dialysis, the dialysis “membrane” is the patient's peritoneal lining (i.e., the serosal membrane covering the bowel). Dialysis solution is pumped into the patient's peritoneal cavity to establish an osmotic and concentration gradient across the peritoneal membrane. This osmotic and concentration differential causes the transfers of undesired materials from the blood into the dialysate and transfers desired materials from the dialysate into the blood. After a prescribed “dwell time,” the dialysate is removed with whatever undesired materials have transferred into it from the blood across the peritoneal membrane and without whatever desired materials have transferred out of it into the blood across the peritoneal membrane. A new solution is then placed into the peritoneal cavity and the process is repeated.

Dialysis is reasonably effective in accomplishing its primary goal of removing toxins and wastes from the bloodstream, but does not address other aspects of deteriorating kidney function. One such aspect involves the serum phosphorus and calcium levels and the hormone from the parathyroid gland (paratharmone or “PTH”). Calcium concentrations in the body are regulated by the kidneys, the parathyroid gland, the gastrointestinal tract and bones, in a complex metabolism. There is an interplay between PTH and a hormone produced by the liver (25 hydroxycholecaliciferol) and converted by healthy kidneys, to 1,25 dihydroxycholecalicefol, the active form of vitamin D. In chronic renal failure, the kidneys convert insufficient 1,25 dihydroxycholecalciferol. In addition, the kidneys do not excrete phosphorus and the 1,25 dihydroxycholecalciferol receptors become passive. All these abnormalities upset the homeostasis of both calcium and phosphate. This process is presented diagramatically in FIG. 1.

The end result in a process not fully understood is insufficient calcium (“hypocalcemia”), excessive phosphorus (hyperphosphatemia”) and overproduction of PTH (“hyperparathyroidism”). The most direct outcome of the abnormalities is bone loss in a condition called “renal osteodystrophy.” The overproduction of PTH (called “secondary hyperparathyroidism” under these circumstances) can produce a variety of ill effects to the organs and tissues. The altered calcium and phosphorus balance is thought to accelerate vascular calcification.

To treat these conditions, dialysis patients are administered supplemental calcium salts or other medications (such as RenalGel) to absorb phosphorous, receive lowered calcium dialysate as well as supplemental vitamin D analogues (1,25 dihydroxycholecalciferol) (such as the brand name Calcijex), paricalcitol (such as the brand name Zempler) or Doxercalciferol (such as the brand name Hectorol). 1,25 dihydroxycholecaliferol and doxercalciferol are available in both the oral and IV forms. Paricalcitrol is available only in the IV form. Data suggests that intravenous administration may be more effective than oral administration. Clinical studies suggest that intravenous Vitamin D decreases the synthesis and release of PTH by the parathyroid gland and increases serum calcium levels. Oral or intravenous administration of 1,25 dihydroxycholecalciferol may accentuate undesirable side effects. 1,25 dihydroxycholecalciferol enhances intestinal absorption of calcium and phosphorus and enhances bone mineral mobilization leading to hyperphosphotemia and hypercalcemia. Paricalcitrol and doxercalciferol are advertised as not absorbing calcium from the intestinal tract to the same degree and have a similar effect on increasing the serum calcium. Consequently 1,25 dihydroxycholecalciferol has been replaced with an analogue that might avoid these ill effects in dialysis patients.

Such an analogue, paracalcitol (19-Nor=−1, 25-(OH)₂D₂), was developed and tested on a limited basis some years ago. Studies in rats demonstrated that paracalcitol suppressed PTH secretion without producing significant hypercalcemia or hyperphosphatemia. See Slatopolsky et al., “A New Analog of Calcitriol, 19-Nor-1, 25-(OH)₂D₂, Suppresses Parathyroid Hormone Secretion in Uremic Rats in the Absence of Hypercalcemia,” American Journal of Kidney Diseases, Vol. 26, No. 5 (November), 1995; pp. 852-860. Later studies found similar results in humans by comparing paracalcitol with calcitriol. See, e.g., Sprague et al., “Suppression of Parathyroid Hormone Secretion in Hemodialysis Patients: Comparison of Paracalcitol with Calcitriol,” American Journal of Kidney Disease, Vol. 38, No. 5, Suppl. 5 (November), 2001; pp. S51-S56. And paracalcitol compared favorably with placebos in other studies. None of these studies examined the effect of paracalcitrol on survival.

Still other studies, however, have been inconclusive. For example, in a “Statistical Review and Evaluation” under NDA #20-819 submitted to the United States Food and Drug Administration, injectable calcitriol (under the brand name Calcijex) was compared with paracalcitol with regard to the incidence of hypercalcemia and elevated Ca X P product level. The results showed that “the incidence of elevated Ca and/or Ca X P levels, as defined in the protocol, was statistically significantly greater in the “paracalcitol patients.”

Paracalcitol is now commonly prescribed in preference to 1,25 dihydroxycholecalciferol for patients with secondary hyperparathyroidism in End Stage Renal Disease. See Llach et al., “Paricalcitol in Dialysis Patients with Calcitriol-Resistant Secondary Hyperparathyroidism,” American Journal of Kidney Diseases, Vol. 38, No. 5, Suppl. 5 (November), 2001; pp. 545-550. Adverse effects reported in the use of paricalcitol, however, include nausea, vomiting, metallic tastes, chills, fever, sepsis, palpitations, dry mouth, gastrointestinal bleeding, edema, light-headedness and pneumonia. See Goldenberg, “Paricalcitol, a New Agent for the Management of Secondary Hyperparathyroidism in Patients Undergoing Chronic Renal Dialysis,” Clinical Therapeutics, Vol. 21, No. 3, 1999. Others have recommended the use of Doxercalciferol which is reported to have similar effects on calcium and phosphorus absorption. Martin K J, Gonzales E A, Vitamin D Analogues for the Management of Secondary Hyperparathyroidism, Am. J. Kidney Dis. 2001; 38 (5 Supp. 5) 534-40.

There has also been considerable uncertainty about the results of all these reported studies since they have involved a relatively modest number of patients. All have a beneficial effect on PTH suppression, but the effects on calcium and phosphorus have been debated. Mortality and hospitalization have not been examined with any of these agents.

### SUMMARY OF THE INVENTION

The invention is a method of reducing mortality in the treatment of chronic renal disease by administering paricalcitol. Exclusive clinical data shows improved survival in dialysis patients treated with paricalcitol as compared with calcitriol, regardless of whether they were hypercalcemic, hyperphosphatemic or hyperparathyroidic. It is unknown whether this effect might be seen in patients with renal failure not yet on dialysis.

Calcitriol therapy may adversely affect patient survival because of widespread cellular and subsequent organ damage. Furthermore, vitamin D receptors are ubiquitous throughout the body, and vitamin D is thought to have effects on inflammation, immune modulation, all growth and cell differentiation. Even slight modifications to the parent active vitamin D 1,25-(OH)₂D₃ can dramatically affect these cellular responses. In contrast to calcitriol, paricalcitol suppresses the vitamin D receptors in the gut and thus it is likely that vitamin D receptors in other organs respond differently.

### BRIEF DESCRIPTION OF THE DRAWINGS

FIG. 1 shows Kaplan-Meier Survival Analysis for Patients Treated with Either Paricalcitol or Calcitrol from 1999 to 2001 (log rank p<0.01).

FIG. 2 shows Hazard Ratios Associated with Paricalcitol Treatment Stratified by Exposure Characteristic in which percent represents fraction of deaths within each strata, boxes represent point estimates, and horizontal lines represent 95% Confidence Intervals.

FIG. 3 shows Hazard Ratios Associated with Quintiles of Serum Calcium, Phosphorus, and Parathyriod Hordmone. HR, hazard ratio; R, reference category; * P<0.05

FIG. 4 shows a diagram involving the homeostasis of calcium and phosphate.

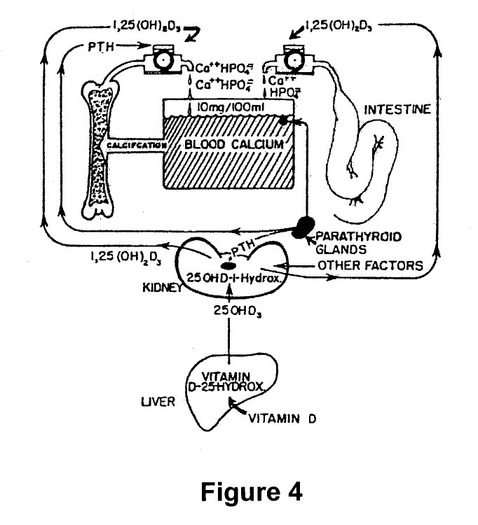

### DETAILED DESCRIPTION OF A PREFERRED EMBODIMENT

An historical cohort study was conducted of patients undergoing chronic hemodialysis in Fresenius Medical Care (FMC) dialysis facilities in the United States. Patients who initiated treatment with either paricalcitol or calcitriol beginning Jan. 1, 1999 or after, and who remained exclusively on that intravenous vitamin D formulation until the conclusion of the follow-up period on Dec. 31, 2001, were included in this study. Patients were excluded if they received any form of intravenous vitamin D prior to Jan. 1, 1999 or if they switched from one injectable vitamin D formulation to another during the study period. Patients treated exclusively with other intravenous vitamin D formulations were not studied because of small sample sizes in those groups. During the study period, the decision to start one formulation of vitamin D over another was made by individual clinicians, FMC had not distributed guidelines to direct injectable vitamin D therapy, and the literature did not provide human data to suggest superiority of one formulation over another with respect to survival.

The FMC data system is an Oracle database populated by the individual clinical data systems employed in each individual FMC dialysis facility. The database contains demographic, laboratory, hospitalization, and mortality information as well as detailed records of the treatments administered during each hemodialysis run since January 1995. All data were collected prospectively as part of routine patient care in over 1000 dialysis facilities throughout the United States. For this study, no additional data were retrospectively abstracted from medical records.

Ascertainment of Exposures, Outcomes and Covariates

Upon a patient's admission to an FMC facility, demographic information including age, gender, race, date of first dialysis, cause of end stage renal disease, and diabetes status were entered into the system. Subsequently, hemodialysis prescription, laboratory tests, and injectable medications were recorded daily. Centralized labs utilized by all FMC facilities performed laboratory tests using standardized assays. Laboratory test results were automatically downloaded from the centralized laboratory to the FMC data system, minimizing the possibility of data entry errors.

Records of all medicines administered during hemodialysis included date of administration, medication name, dose, and route of administration. This information was collected and uploaded into a central database on a daily basis, and underwent routine quality assessment and control measures because of their link with billing systems. This permitted restriction of the analysis to those who initiated and remained on a single vitamin D formulation during the study period. Whenever a patient missed a hemodialysis treatment, a temporary absence or permanent discharge must be recorded in the system by the facility staff in order to complete the daily reconciliation of prescribed verses administered treatments. Therefore, all patient deaths, including date and cause of death (ICD-9 coded), were recorded in the database by the individual facilities as one type of permanent discharge. In addition, all hospitalizations are recorded as temporary absences, even if no dialysis treatment was missed. Data entered by the individual facilities underwent continuous quality improvement assessment to ensure their accuracy and completeness.

The base line for each individual patient was defined as within a three-month period before starting on paricalcitol or calcitriol. Base line laboratory values were obtained by averaging all values in the three months prior to initiating vitamin D therapy. Quintiles of base line serum calcium and phosphate levels were determined by aggregating values of all patients in the three year time period. Because of known lot-to-lot drifts in parathyroid hormone (PTH) assays, serum levels of PTH were categorized by yearly quintiles, and comparative quintiles across years were combined for the analysis.

Patient “vintage” was determined as the number of days from the initiation of chronic hemodialysis to the first day paricalcitol or calcitriol was administered. This covariate was examined both as a continuous and a categorical variable. As a measure of unknown confounders related to facility-specific practices, the standardized mortality rate (SMR) for each facility was calculated. The SMR is a facility-specific mortality rate relative to all of the FMC centers throughout the United States, and adjusts for between dialysis center variations in survival that are beyond typical explanatory variables such as differences in nutrition, degree of anemia, and measures of dialysis adequacy. As a measure of unknown confounding related to natural improvements in clinical practice over time, study entry period, defined as the calendar quarter in which a patient started vitamin D treatment, also was included in the analysis.

Patients were analyzed according to the vitamin D formulation they had initiated on or after Jan. 1, 1999. Standard univariate (Chi square and t-tests) analyses were performed, and means, standard deviations (SD), and interquartile ranges (IQR) were used for descriptive purposes. Mortality rates according to vitamin D formulation were calculated by dividing the number of subjects who died in the follow-up period by the number of person-years of observation contributed by the subjects. The Kaplan-Meier method was used to examine crude survival analysis, and Cox proportional-hazards regression analysis was used to adjust for potential confounders. Patients who left their FMC facility or underwent kidney transplantation were censored. Hazard ratios for mortality, with 95% confidence intervals, were calculated for patients treated with paricalcitol—patients treated with calcitriol served as the reference category in all analyses except when otherwise specified. Cox models adjusted for potential confounding variables also were used to examine stratum specific hazard ratios associated with paricalcitol treatment. Base line hospitalization frequency before initiating paricalcitol or calcitriol was compared, as were major causes of mortality (infection, neoplasm, cardiovascular, cerebrovascular, and other) after starting paricalcitol or calcitriol. Finally, treatment-specific hazard ratios were calculated according to quintiles of base line serum calcium, phosphate, and parathyroid hormone levels adjusted for potential confounders. This analysis was performed to uncover potential non-linear trends, and to determine if differences in risk exist between the vitamin D formulations. Analyses were performed with SAS software (SAS Institute, Cary, NC). All P values were two sided, and P values less than 0.05 were considered to indicate statistical significance.

During the 36 months of follow-up, 27,398 chronic hemodialysis patients initiated and remained on paricalcitol, and 23,516 on calcitriol. The base line characteristics (in Table 1 below) suggested patients receiving paricalcitol were younger (interquartile range (IQR), paricalcitol 50-73 years; calcitriol 52-75 years), more likely to be African American, and more likely to have arteriovenous fistulae for their vascular access. The paricalcitol group also had higher base line serum levels of calcium, phosphate, calcium-phosphate product, and parathyroid hormone. Patients selected for paricalcitol treatment tended to be larger than patients selected for calcitriol treatment and had slightly higher concentrations of serum albumin and creatinine. Base line measures of dialysis adequacy, however, were similar between the two groups. The number of days between dialysis initiation and start of either paricalcitol or calcitriol (vintage days) was longer for paricalcitol, (612±1037 days, IQR 30-763 days) than calcitriol (489±965 days, IQR 21-495 days, p<0.01). Crude hospitalization rates within one-year prior to start of vitamin D formulation were similar (28.1% paricalcitol, 27.5% calcitriol, p=0.15), as were the mean standardized mortality rates associated with dialysis facilities patients underwent hemodialysis (1.10 paricalcitol, 1.09 calcitriol, p=0.77).

TABLE 1

| | Paricalcitol | Calcitriol | |
|---|---|---|---|
| Characteristic | N = 27,398 | N = 23,516 | |
| Age (years) | 61 | 63 | <0.01 |
| Gender (% male) | 53 | 54 | 0.01 |
| Race (%) | | | <0.01 |
| Caucasian | 53 | 58 | |
| African-American | 38 | 33 | |
| Other | 9 | 9 | |
| Diabetes (%) | 48 | 51 | <0.01 |
| Vascular Access | | | <0.01 |
| Fistula (%) | 21 | 18 | |
| Graft (%) | 27 | 26 | |
| Catheter (%) | 23 | 26 | |
| Body Mass Index (kg/m²) | 28.6 ± 8.6 | 28.2 ± 9.1 | <0.01 |
| Body Surface Area (m²) | 1.9 | 1.8 | <0.01 |
| Albumin (g/dl) | 3.7 ± 1.0 | 3.6 ± 0.5 | <0.01 |
| Calcium (mg/dl) | 8.7 ± 0.8 | 8.5 ± 0.9 | <0.01 |
| Phosphorus (mg/dl) | 5.6 ± 1.6 | 5.3 ± 1.5 | <0.01 |
| Calcium X Phosphorus (product) | 48 ± 15 | 45 ± 14 | <0.01 |
| Parathyroid Hormone (pg/ml) | 493 ± 359 | 389 ± 308 | <0.01 |
| Alkaline phosphatase (U/L) | 127 ± 90 | 130 ± 103 | <0.01 |
| Hemoglobin (g/dl) | 10.8 ± 1.5 | 10.7 ± 1.6 | <0.01 |
| Ferritin (ng/ml) | 382 ± 422 | 370 ± 438 | 0.01 |
| White Blood Cell Count (per mm³) | 8 ± 3 | 8 ± 3 | ns |
| Bicarbonate (mmol/L) | 21 ± 4 | 20 ± 4 | <0.01 |
| Creatinine (mg/dl) | 7.8 ± 3.1 | 7.5 ± 3.1 | <0.01 |
| URR † (%) | 68 ± 9 | 67 ± 10 | ns |

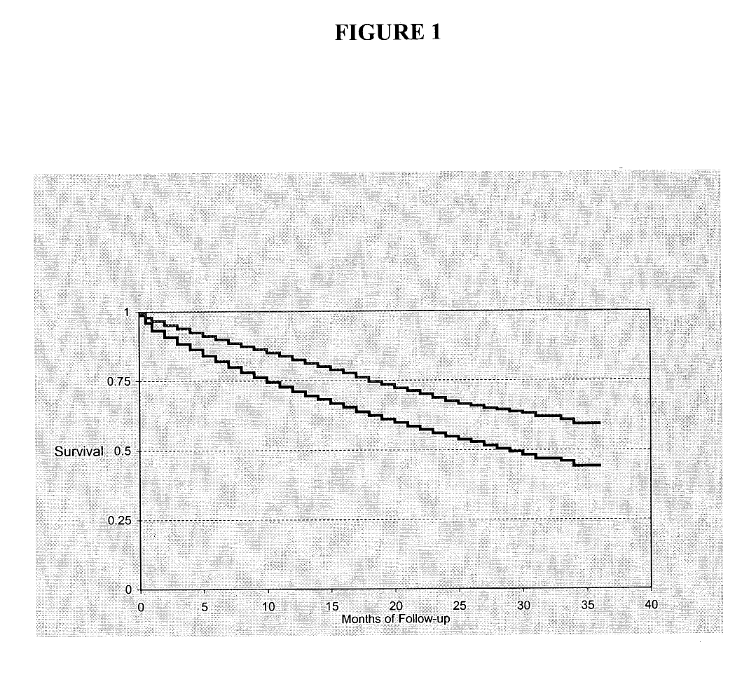

During the 36-month follow-up period after the initiation of injectable vitamin D therapy, 10,222 of the 50,916 (20%) patients died. Mortality rates significantly differed between the two groups: 3417 deaths/18,430 person-years (18.54%) in the paricalcitol group, compared with 6805 deaths/22,057 person-years (30.85%) in the calcitriol group (Rate Ratio 0.60, 95% CI 0.58-0.63, p<0.01). Crude survival for the entire cohort according to treatment status was then examined (see FIG. 1). Survival at one year was 82.6% among patients treated with paricalcitol, compared with 73.7% for those receiving calcitriol. At two years, crude survival was 68.6% paricalcitol and 56.0% calcitriol, and at three years, 59.3 and 44.1%, respectively. Examination of mortality by ICD-9 codes demonstrated paricalcitol treatment was associated with a greater than 50% risk reduction (p<0.01) for each cause (infection, neoplasm, cardiovascular, cerebrovascular, and other), with no specific cause predominating.

Cox proportional-hazards regression analysis was performed to investigate whether confounding covariates could explain the results (see Table 2 below).

TABLE 2

| Models | N | HR | 95% CI | P value |
|---|---|---|---|---|
| Unadjusted | 50,916 | 0.58 | 0.55-0.60 | <0.01 |
| Case-Mix † | 50,572 | 0.61 | 0.59-0.64 | <0.01 |
| Case-Mix † and Study Entry | 50,572 | 0.70 | 0.67-0.73 | <0.01 |
| Period | | | | |
| Case Mix †, Study Entry Period, | 50,572 | 0.69 | 0.66-0.73 | <0.01 |
| SMR ‡ | | | | |
| Case Mix †, Study Entry Period, | 50,572 | 0.70 | 0.67-0.74 | <0.01 |
| SMR ‡, Dialysis Access | | | | |
| Case Mix †, Study Entry Period, | 25,471 | 0.73 | 0.69-0.78 | <0.01 |
| SMR ‡, Dialysis Access Base-Line | | | | |
| Laboratory Values § | | | | |

Compared to the unadjusted model, the point estimate changed when adjusted for case-mix variables including age, gender, race, diabetes, and vintage. The next appreciable change in point estimates was noted when the model included study entry period. Because a potential survival benefit may exist for those entering the study at later time periods, adjusting for study entry period reduced the point estimate but did not extinguish the effect. Thereafter, with the addition of other potential confounders, including adjustment for base line laboratory values, point estimates did not appreciably change but confidence intervals widened expectedly. Nonetheless, while progressive adjustments reduced the apparent risk benefit associated with paricalcitol treatment from approximately 42% to 27%, the benefit could not be extinguished and remained robust over all analyses. Covariates in the final model (n=25,471) and their respective hazard ratios are shown in Table 3.

TABLE 3

| Characteristic | X² | P value | | 95% CI |
|---|---|---|---|---|
| Paricalcitol (Ref = calcitriol) | 81 | <0.01 | 0.733 | 0.686-0.784 |
| Age (years) | 417 | <0.01 | 1.024 | 1.022-1.026 |
| Gender (Ref = female) | 107 | <0.01 | 1.397 | 1.311-1.488 |
| Race (Ref = non-white) | 2 | 0.18 | 1.044 | 0.980-1.112 |
| Diabetes (Ref = no) | | | | |
| Yes | 34 | <0.01 | 1.205 | 1.132-1.283 |
| Unknown | 0 | 0.59 | 1.033 | 0.919-1.160 |
| Vintage (√days) | 104 | <0.01 | 1.011 | 1.009-1.013 |
| Standardized Mortality Rate | | | | |
| (Ref = Medium) | | | | |
| High | 131 | <0.01 | 1.435 | 1.349-1.526 |
| Low | 43 | <0.01 | 0.758 | 0.698-0.823 |
| Vascular access (Ref = fistula) | | | | |
| Graft | 64 | <0.01 | 1.482 | 1.345-1.633 |
| Catheter | 408 | <0.01 | 2.688 | 2.442-2.959 |
| Unknown | 2 | 0.17 | 0.932 | 0.842-1.031 |
| Body Surface Area (m²) | 64 | <0.01 | 0.589 | 0.517-0.670 |
| Albumin (g/dl) | 237 | <0.01 | 0.587 | 0.548-0.628 |
| Calcium (mg/dl) | 21 | <0.01 | 1.101 | 1.056-1.148 |
| Phosphorus (mg/dl) | 52 | <0.01 | 1.086 | 1.062-1.111 |
| Parathyroid Hormone (pg/ml) | 2 | 0.16 | 1.000 | 1.000-1.000 |
| Alkaline phosphatase (U/L) | 65 | <0.01 | 1.001 | 1.001-1.001 |
| Hemoglobin (g/dl) | 44 | <0.01 | 0.933 | 0.914-0.952 |
| White Blood Cell Count (per mm³) | 28 | <0.01 | 1.020 | 1.013-1.028 |
| Ferritin (ng/ml) | 38 | <0.01 | 1.000 | 1.000-1.000 |
| Bicarbonate (mmol/L) | 4 | 0.05 | 0.911 | 0.982-1.000 |
| S-GOT (U/ml) | 65 | <0.01 | 1.001 | 1.001-1.001 |
| Creatinine (mg/dl) | 80 | <0.01 | 0.938 | 0.925-0.951 |

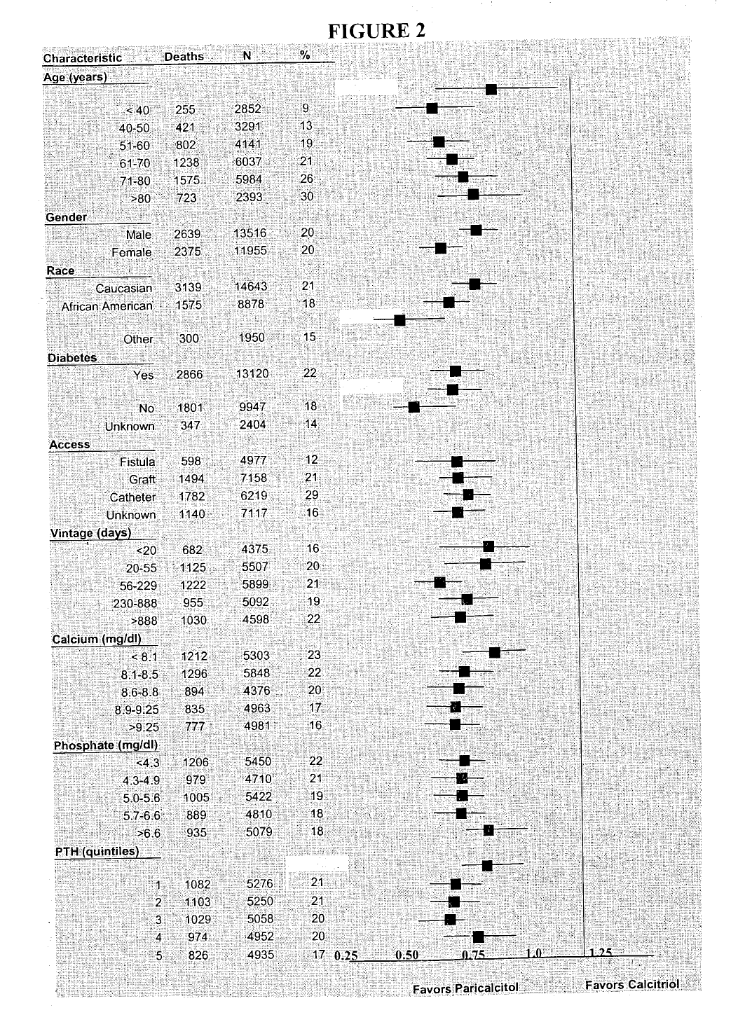

Formal testing for effect modification did not reveal that the effect of treatment on survival varied with any of the covariates tested. To investigate the possibility of residual confounding, however, the hazard ratios associated with paricalcitol treatment in multiple strata adjusted for potential confounding covariates was examined (see FIG. 2). Only patients less than 40 years of age at the time of starting injectable paricalcitol did not demonstrate a significant survival advantage over similar age patients starting on calcitriol. This was also the group with the lowest event rate (9%). In all other strata, including those who began paricalcitol therapy within 20 days of chronic hemodialysis initiation and in all strata of base line calcium, phosphate, and parathyroid hormone (PTH) levels, hazard ratios approximated the overall hazard ratio (Table 2) associated with paricalcitol treatment. Imposing multiple restrictions to the study population, therefore, was not expected to alter the results. For example, restricting the analysis to Caucasian diabetic patients, ages 60-70 years, with a vintage date<100 days, and arteriovenous prosthetic graft for access, the unadjusted (HR 0.53, 95% CI 0.33-0.85) and adjusted (HR, 0.42, 95% CT 0.21-0.82) benefit of paricalcitol remained significant.

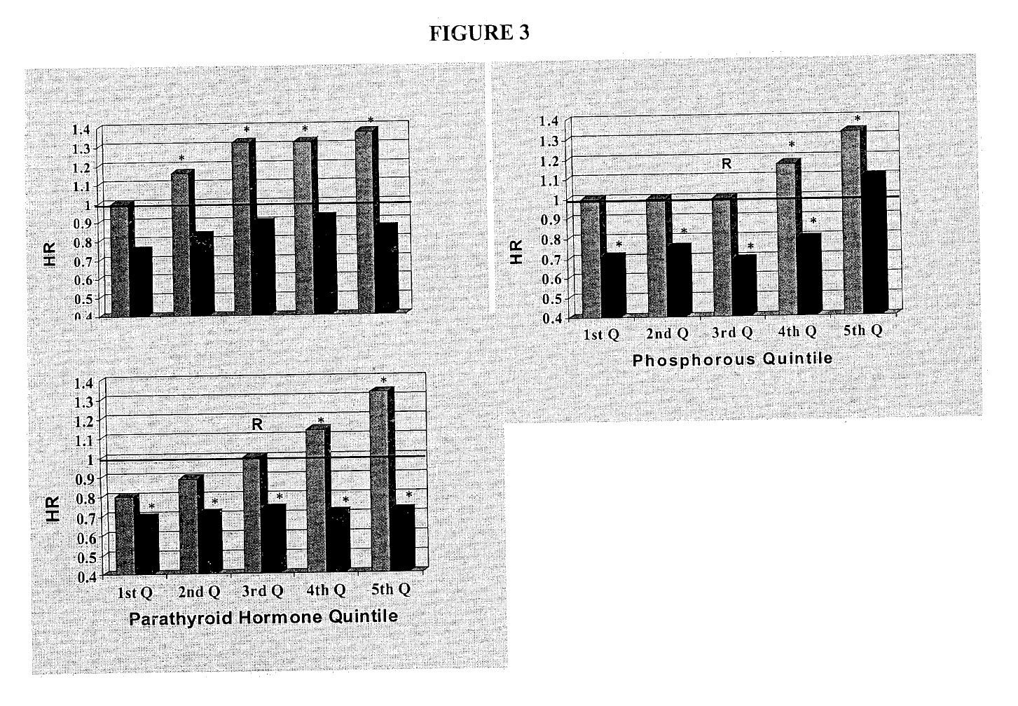

Mortality was examined according to base line measurements of calcium, phosphate, and PTH differed between the two groups (see FIG. 3). In this analysis, the hazard ratios associated with specific quintiles of each covariate were determined according to injectable vitamin D formulation. The final model was adjusted for all covariates (as shown in Table 3 above) and also consisted of nine (n−1) treatment X quintile covariates. While the mortality risk increased with each successive quintile of base line serum calcium among those treated with calcitriol, paricalcitol treated patients did not appear to have an increased risk of mortality regardless of base line serum calcium level. The mortality risk increased with successive quintiles of serum phosphorus regardless of injectable vitamin D formulation, but within each quintile the mortality risk was comparatively lower among the paricalcitol group compared with the calcitriol group. The observed mortality risk according to quintiles of PTH levels followed a similar pattern as that for serum calcium: while the mortality risk increased with each successive quintile of base line PTH among patients treated with calcitriol, paricalcitol treated patients had a significantly lower risk of mortality at all levels of PTH, and this survival benefit did not appear to diminish even in the highest quintile of PTH. Finally, two separate multivariable analyses stratified by vitamin D formulation were performed to examine the association between quintiles of calcium, phosphate, and PTH and mortality within each group of vitamin D formulation and similar results were found (data not shown) as those observed above.

In this historical cohort study of hemodialysis patients who initiated intravenous vitamin D therapy between 1999 and 2001, patients treated with paricalcitol had a significant survival advantage compared to those treated with calcitriol. This survival advantage was evident within the first year of starting paricalcitol, and continued to increase in the ensuing 36-month follow-up period. The survival advantage observed was independent of baseline calcium, phosphorus, or parathyroid hormone (PTH) levels, and other potential confounding laboratory and demographic characteristics. Furthermore, in stratified analyses, the benefit of paricalcitol remained significant in almost every strata of age, and in all other strata including gender, race, diabetes status, duration of dialysis before starting paricalcitol, and in all strata of base line serum calcium, phosphorus, and PTH. These results suggest that paricalcitol should be preferred over calcitriol when the decision is made to initiate injectable vitamin D therapy for management of secondary hyperparathyroidism among chronic hemodialysis patients.

Secondary hyperparathyroidism has been extensively studied in patients with end-stage renal disease. While secondary hyperparathyroidism is the leading cause of skeletal disease among patients with end-stage renal disease, recent evidence suggests hyperparathyroidism also contributes to arterial wall thickening and calcification, hypertension, myocardial fibrosis, dyslipidemia, and increased mortality among dialysis patients. Reduced renal 1-hydroxylation of 25-OH-cholecalciferol to its active form impairs intestinal calcium absorption, leading to hypocalcemia and compensatory increase in PTH secretion. Impaired excretion of phosphate by the end-stage kidney leads to hyperphosphatemia, which further stimulates PTH secretion. The inability of the kidney to increase active vitamin D levels in response to PTH leads to ongoing bone resorption and release of PTH from a lack of feedback inhibition by vitamin D, further increasing PTH levels. Calcium supplementation, dietary phosphate restriction, and oral phosphate binders are first-line therapies, but despite this, up to 60% of patients eventually require intravenous vitamin D therapy to control PTH secretion and maintain normal serum calcium levels. Because therapy with the standard active vitamin D calcitriol also stimulates gut mineral absorption and can lead to hypercalcemia and hyperphosphatemia, clinicians are forced to continually balance the need for PTH suppression with altered mineral metabolism. Given the association between hyperparathyriodism, hyperphosphatemia, and elevated calcium-phosphate product with increased morbidity and mortality among chronic dialysis patients, and that the two formulations of injectable vitamin D we studied likely have differing effects on these parameters, significant differences were found in survival according to which formulation of vitamin D a patient had received.

The study design employed was a historical cohort study in which patients were selected based on prior exposure to an injectable vitamin D formulation, and outcomes (deaths) already had occurred prior to initiating this study. While the usual limitations of retrospective analysis, including selection bias, cannot completely be excluded, the data were strengthened by their prospective collection, comparison of contemporaneous groups in similar dialysis facilities, and the inclusion of all patients naive to injectable vitamin D at the time of entry into the study. In addition, the large number of patients examined in this study minimizes significant bias that may have been introduced by practice variations from a limited number of facilities. Importantly, because of their link to the centralized database used by all individual dialysis facilities in the Fresenius Medical Care network, the primary exposures in this study, treatment with injectable paricalcitol or calcitriol, and the primary outcome, survival, were well documented. In addition, analyses were restricted to patients who remained on one formulation of injectable vitamin D for the entire duration of the follow-up, reducing the possibility of misclassification of the primary exposure. Finally, all covariates important to include in the multivariable models including race, diabetes status, vintage date, study entry period, and an array of laboratory variables were collected prospectively and entered into the central data base while these patients were undergoing routine chronic hemodialysis, and thus retrospective abstraction of such information from medical records was unnecessary.

Prior to this study, no outcome data in humans were available to suggest a survival benefit of paricalcitol over calcitriol. Nonetheless, the possibility cannot be excluded that individual nephrologist's selection of paricalcitol or calcitriol was linked to other potential confounding factors that were also linked to the outcome. The possibility that such non-random assignment of therapy could have led to unequal susceptibility to the outcome is a criticism of observational studies that only true randomization can ameliorate. From Table 1, there appeared to be selection bias in favor of patients treated with paricalcitol. Indeed, adjustment for these and other measures reduced the apparent survival benefit associated with paricalcitol treatment from 42% to 27%. Although vintage differed between the groups, which possibly conferred a healthy survivor advantage to the paricalcitol group, adjustment for vintage in the multivariable analysis did not appreciably change the effect size. In addition, in the stratified analysis when strata of different vintage periods were analyzed separately, paricalcitol treatment was associated with a significant survival benefit irrespective of vintage. Base line levels of specific minerals including serum phosphorus and calcium were higher among those treated with paricalcitol, and the association between hyperphosphatemia and elevated calcium-phosphate product with increased vascular calcification and mortality among dialysis patients would argue that this group started with a survival disadvantage. Nonetheless, the benefit cannot be extinguished, and the residual benefit was not trivial. Importantly, in addition to adjusting for potential covariates that might have affected nephrologists' choice, the final model also included covariates that accounted for a possible learning curve that might be expected with the introduction of a new drug. Stratified models were analyzed to determine if specific patient characteristics accounted for the observed effect, and when benefit is observed across several strata, the argument that the benefit is attributable to the intervention and not to inequalities in specific subgroups is strengthened. In all strata examined except for patients less than 40 years old, the findings remained significant. In fact, the magnitude of effect was similar across all strata, suggesting the benefit of paricalcitol is generalizable to a diverse group of hemodialysis patients. Not surprisingly, for example, when the analysis was restricted to individuals meeting five entry criteria (e.g., age 60-70 years, Caucasian, presence of diabetes, vintage<100 days, and prosthetic graft for vascular access) the benefit of paricalcitol treatment remained significant. In the single strata that yielded a non-significant finding (age<40 years), the event rate was low. The possibility that hemodialysis patients below 40 years of age may not demonstrate a survival benefit from paricalcitol treatment, however, cannot be excluded.

Incomplete information regarding oral medication use is an important limitation of this study. Oral vitamin D is commonly used among patients with end-stage renal disease, but when injectable vitamin D is initiated, oral formulations are usually discontinued. Therefore, oral vitamin D intake likely did not contribute to the findings. Accurate information on the use of calcium-based (e.g. calcium acetate or carbonate) versus non-calcium based (e.g., sevelamer) phosphate binders was also unavailable. There is a suggestion that sevelamer, for example, is associated with reduced vascular calcification compared with calcium-based binders. Nevertheless, this medication did not likely explain the findings since national sevelamer use was only ˜10% by the end of 1999, 20% by the end of 2000, and ˜30% by the end of 2001 (http://www.imshealth.com), and the results remain robust even when each year was analyzed separately (data not shown). In addition, because calcitriol use is more commonly associated with hypercalcemia and hyperphosphatemia than paricalcitol, sevelamer would have more likely been prescribed to patients taking calcitriol, reducing the possibility that this medication could account for the survival advantage of paricalcitol. Finally, paricalcitol treatment was beneficial even in the lowest strata of base line calcium and phosphorus, the patient group least likely to receive sevelamer.

In vitro, calcitriol sensitizes cells to ATP-depletion and iron-mediated injury when compared with paricalcitol, and these changes are evident independent of changes in levels of serum calcium, phosphate, and PTH. This latter finding supports an earlier observation that 1 alpha-hydroxyvitamin D₂ compounds are 5 to 15 times less toxic than 1 alpha-hydroxyvitamin D₃ compounds in animals. Therefore, it is possible that calcitriol therapy adversely affected patient survival because of widespread cellular and subsequent organ damage. Furthermore, vitamin D receptors are ubiquitous throughout the body and vitamin D is thought to have effects on inflammation, immune modulation, cell growth, and cell differentiation. Importantly, slight modifications to the parent active vitamin D 1,25-(OH)₂D₃ can dramatically affect these cellular responses. In contrast to calcitriol, for example, paricalcitol suppresses the vitamin D receptors in the gut, and thus it is likely that vitamin D receptors in other organs respond differently to the two formulations. Furthermore, because paricalcitol appears to be less effective in gut absorption and bone reabsorption of minerals, calcium and phosphate loads may have differed between the two groups, which could have increased the risk for vascular calcification and cardiovascular related mortality. Finally, in this study, mortality risk among those receiving calcitriol increased with successive increases in base line levels of serum calcium and parathyroid hormone, whereas the risk remained comparatively lower in all paricalcitol groups. In fact, the relative benefit of paricalcitol was not attenuated at any level of serum calcium or PTH. Therefore, paricalcitol may have been acting independently of serum calcium and PTH, or alternatively, paricalcitol may have been affecting the PTH-calcium axis differently than calcitriol.

Although treatment with paricalcitol was unable to completely ameliorate the increased mortality risk associated with the highest base line levels of serum phosphorus (>6.6 mg/dl), at all levels of serum phosphorus paricalcitol treatment still conferred a survival advantage over calcitriol treatment. The association between elevated serum phosphorus and increased mortality among chronic dialysis patients has been well documented, however the exact mechanism underlying this observation is less clear. When causes of mortality were examined, hyperphosphatemia was associated with an increased risk of mortality from a variety of cardiovascular and non-vascular causes including infection. In this current study, paricalcitol treatment was associated with a reduction in mortality from all the major causes examined (cardiovascular, cerebrovascular, neoplastic, infections), and no one etiology predominated. One explanation for this may have been our reliance on ICD-9 codes, whose accuracy has been questioned because of their strong influence by reimbursement mechanisms, and because ICD-9 codes were not validated. Alternatively, just as has been speculated with hyperphosphatemia, given that cardiovascular disease is the most common cause of mortality among dialysis patients, a cardiovascular mechanism is probable. The finding that treatment with paricalcitol attenuated the mortality risk at all levels of serum phosphorus and at least partially had an impact at the highest base line level does suggest that a phosphate-related mechanism warrants further investigation.

What is claimed is:

1. A method for reducing mortality in a renal failure patient by administering paricalcitol in conjunction with dialysis treatment.

2. The method of claim 1, wherein said step of administering paricalcitol is without regard for whether the patient has secondary hyperparathyroidism.

3. The method of claim 1, wherein said step of administering paricalcitol is without regard to whether the patient is hypercalcemic or hypocalcemic.

4. The method of claim 1, wherein said step of administering paricalcitol is without regard to whether the patient is hyperphosphatemic or hypophosphatemic.

5. The method of claim 1, wherein said method does not include administering calcitriol.
